(19) United States
(12) Patent Application Publication
(10) Pub. No.: US 2017/0268996 A1
(43) Pub. Date: Sep. 21, 2017

(54) METHOD AND DEVICE FOR MEASURING THE GAS CONTENT OF MATERIALS PACKAGED IN PLASTIC FILMS, GLASS OR OTHER LIGHT-PERMEABLE MATERIALS AND SENSITIVE TO A GAS TO BE MEASURED

(71) Applicant: TecSense GmbH, Grambach (AT)

(72) Inventors: Johannes KROTTMAIER, Graz (AT); Volker RIBITSCH, Graz (AT)

(73) Assignee: TecSense GmbH, Grambach (AT)

(21) Appl. No.: 15/309,830

(22) PCT Filed: Apr. 29, 2015
(86) PCT No.: PCT/AT2015/000060

§ 371 Date: Nov. 9, 2016

(30) Foreign Application Priority Data
May 14, 2014 (AT) GM 207/2014

(51) Int. Cl. G01N 21/64; G01N 33/00; G01N 33/12

(52) U.S. Cl. CPC G01N 21/64; G01N 33/12; G01N 33/0036

(57) ABSTRACT

A method for the non-invasive measurement of the gas content of at least one gas selected from O₂, CO₂, amines, nitrogen oxides, sulfur compounds, in transparent packages made of plastic films, glass or other light-permeable materials, in which a sensor material sensitive to the gas content in the interior of the package is excited by excitation light incident thereon to emit a fluorescent or luminescent light, the emitted fluorescent or luminescent light is detected in a detection device interacting with the sensor, whereupon the packaged materials or produced materials are subjected to sorting, and optionally counting, as a function of the gas content determined by an evaluation unit coupled to the detection device, comprising the steps of:

a) optionally deep-drawing the package;

b) applying a sensor material to the inner side of a cover for the package, or to the inner side of the package, and optionally drying the sensor material;

c) introducing into the package the materials to be packaged;

d) gas-tightly closing the package by the cover while introducing a modified atmosphere of the gas to be measured;

e) placing the package on a conveying and sorting device;

f) exciting the sensor material by at least one excitation light source arranged at a defined distance from the package;

g) detecting, in a contactless manner, the intensity of the fluorescent or luminescent light emitted by the sensor material as a function of the gas content in the interior of the package, by a detection device arranged at a defined distance from the conveying device during conveying of the package to the sorting device;

h) electrically or electronically converting in the evaluation unit a measurement value detected in the detection device;

i) optionally applying a pressure to the package, and performing steps t) to h) again;

j) outputting a confirmation signal or a rejection signal by the evaluation unit; and

k) forwarding the package to further processing or disposal as a function of the gas content in the package calculated by the evaluation unit, and a device therefore. (FIG. 1)

### BACKGROUND OF THE INVENTION

The present invention relates to a method for the non-invasive measurement of the gas content of at least one gas selected from O₂CO₂, amines, nitrogen oxides, sulfur compounds, in transparent packages made of plastic films or other light-permeable materials, with the proviso that the materials are deformable under pressure, in which a sensor material sensitive to the gas content in the interior of the package is excited by excitation light incident thereon to emit a fluorescent or luminescent light, the fluorescent or luminescent light is detected in a detection device interacting with the sensor, whereupon the packaged materials or produced materials are subjected to sorting, and optionally counting, as a function of the gas content determined by an evaluation unit coupled to the detection device.

Known measuring methods for measuring the gas contents of materials packaged in containers permeable to excitation light, which contain materials sensitive to a gas to be measured, such as oxygen, nitrogen oxides, ammonia or the like, or even form such gases as decomposition products, are currently checked for the content of the interesting gas by non-continuously operating methods. On the one hand, such methods are slow and, on the other hand, they can only be performed on random samples of the packaged materials because of the necessity to examine every sample individually and separately, or a whole batch including a larger number of individual products is collectively examined. With foods and on the pharmaceutical sector, in particular, it is necessary to check whether products comply with the strict standards required, and in case a faulty product is found, the whole batch must usually be sorted out, since it is not known whether, and how many, other samples are likewise defective or have gas contents exceeding defined limit values.

From EP 2 256 042 A1, a packaging machine writ a gas concentration measuring device has become known, in which a measuring device is provided, which comprises a measuring head for reading an indicator for the concentration of the gas by means of electromagnetic radiation, which indicator is arranged in the interior of the chamber, in the interior of the packaging and/or in the interior of a gas line. Such a device enables the measurement of the gas content of a packaging without having to take a sample therefrom.

From US 2007/212789 A1, a portable measuring device can be taken, by which a luminescent compound provided in the interior of a package can be read out so as enable the measurement of the oxygen concentration inside the package as a function of the temperature.

From U.S. Pat. No. 5,407,829 A, a non-contact sensor for measuring the gas content in the interior of a package can be taken.

Another important criterion in the examination of materials with respect to a desired gas content consists in that measurements are able to be per non-invasively, since otherwise this would involve the risk of contamination with harmful substances or of the packages being damaged after an optionally invasively performed method such that the materials contained therein would again have to be discarded after testing. Also in this case, there is the problem of having to reject the whole batch rather than only the examined package if too high a content of the gas to be examined is contained in the package, since an examination of all samples does not seem possible from an economic point of view.

The term “materials” contained in packages as used herein is not limited to a specific state of matter of the contents of packages, but comprises solid, semi-solid and liquid materials as well as gases dissolved in liquids, in particular liquids comprising the gas to measured in a dissolved state. Furthermore, the term material is not limited to foods or pharmaceuticals but may also comprise analytes or, for instance, storage and/or cleaning fluids.

### BRIEF SUMMARY OF THE INVENTION

The present invention aims to provide a non-invasive measuring method by which it is possible to continuously examine any materials of similar type packaged in plastic films or other light-permeable materials, and to selectively sort out defective products or products having too high a content of the gas to he examined, without being forced to reject the whole batch and without causing damage to the packaging or the packaged product during examination.

To solve this object, the method according to the invention is essentially characterized in that the following steps are performed:

a) optionally deep-drawing the package;

b) applying a sensor material to the inner side of a cover for the package, or to the inner side of the package, and optionally drying the sensor material;

c) introducing into the package the materials to be packaged;

d) gas-tightly closing the package by the cover while introducing a modified atmosphere of the gas to be measured;

e) placing the package on a conveying and sorting device;

f) exciting the sensor material by at least one excitation light source arranged at a defined distance from the package;

g) detecting, in a contactless manner, the intensity of the fluorescent or luminescent light emitted by the sensor material as a function of the gas content in the interior of the package, by a detection device arranged at a defined distance from the conveying device during conveying of the package to the sorting device;

h) electrically or electronically converting in the evaluation unit a measurement value detected in the detection device;

i) applying a pressure to the package;

j) performing steps f) to h) again;

k) outputting a confirmation signal or a rejection signal by the evaluation unit; and

l) forwarding the packages to further processing or disposal as a function of the gas content in the packages calculated by the evaluation unit.

In that the method according to the invention is conducted such that a sensor material is applied to an inner side of a cover for a package and said sensor material, required, is subjected to drying, it has become possible to provide a sensor in direct contact with the materials to be examined and contained in a package, which sensor after the gas-tight closure of the package can be excited by an excitation and detection unit to emit fluorescent or luminescent light directly on a conveying and sorting device, and the emitted fluorescent or luminescent light can be read out. The excitation and detection unit in this case comprises, disposed in one- and the same component, at least one excitation light source exciting the sensor material to emit fluorescent or luminescent light as a function of the gas content in the interior of the package, and a detection device arranged on a common carrier together with the at least one excitation light source, which detection device is arranged at a defined distance from the conveying device and forms a detection and evaluation unit, in particular together with the excitation light source and a measuring and evaluation unit. After the reception of a respective fluorescent or luminescent signal and calculation of a measurement value, said measurement value is subsequently electronically or electrically converted in the detection device, and a confirmation or a rejection signal is emitted by the evaluation unit. If a confirmation signal is received, i.e. the gas content inside the package is within predefined limits, the sensitive material packaged in a plastic film or any other light-permeable material is forwarded by the conveying and sorting device to further processing such as further packaging in large containers or to immediate consumption, or to disposal in the case of a rejecting signal. Such a method, therefore, not only enables the non-invasive examination of each individual product, but the method is operated at rates that enable the measurement to be directly performed on a without having to subject the products or packaged materials to further, separate detection steps, thus providing a particularly efficient and time-saving method for determining the gas content in the interior of a package.

According to the invention, a pressure is, moreover, exerted on the cover of the package after the gas-tight closure of the package such that, in the case a leakage or incomplete sealing of the package, gas/air will flow into the interior of the package after having released the pressure on the package. The gas tightness can thus be safely and reliably determined in a subsequently performed, second measurement of a fluorescent or luminescent signal, since a measuring signal corresponding to a gas content that differs from the first measurement value will be obtained. A leaking package can thus be located without having to wait, for instance, until a shift in the concentration of the individual remainders of gases inside the package has occurred by penetration through a small leakiness or by the biological decomposition of the products contained in the interior of the package.

The term gas content in the context of the present application is considered to be synonymous with the term concentration of the respective gas.

According to a further development of the method, the sensor material is applied to the cover of the package by application methods known per se, such as pad printing, screen printing, offset printing, gravure printing, brushing or sputtering, or even labeling. All of the application methods are known per se, wherein it is essential in the context of the method according to the invention that a continuous application method is chosen, by which sufficiently thin or small sensors can be applied to the package and rapid drying of the sensor material is possible in order to not interrupt the continuous packaging of materials to be examined, such as food products or drugs, and, at the same time, also provide a sensor for the safe and reliable detection or the gas content in the interior of the package without causing damage to the packaged material or the package itself.

According to a further development of the invention, a sensor material reacting to O₂; CO₂; NH₃, amines or NO₂ is applied as sensor material to the cover, in particular its inner side, which will subsequently be in contact with the materials to be examined so as to ensure the safe and reliable measurement of the gas content in the interior of the package.

For a precise and, in particular, reversible measurement of the gas content inside the package and, in particular, in order to measure whether a content of the as to be examined exceeds a limit value admissible in the package, a modified atmosphere of the gas to be measured is fed to the package prior to measuring and during closure. Such a procedure ensures that, on the one and, it can be ascertained whether the closure is gas-tight and, on the other hand, it can be determined whether products contained in the interior of the package give off the gases to be examined during their decomposition or destruction, in which case the products will again have to be disposed of.

It goes without saying that the gas content can also be read out at a later point of time, for instance after a given period of storage, in order to determine whether a leakage or a degradation or decomposition of the packaged product has occurred.

In order ensure continuous operation, the method is substantially conducted in such a manner that the measurement of the gas content is performed by the serial excitation and detection of the fluorescent or luminescent light at constant time intervals of 20 ms to 80 ms, in particular 50 ms. Such a procedure ensures that that a plurality of measured values are generated, and moreover safeguards that each package and the materials contained therein or introduced thereinto are safely and reliably measured, whereupon it is possible to determine by the evaluation of the obtained measurements whether or not a packaged product or the package, or the container itself correspond to the quality requirements.

An correspondence with a further development of the invention, measurement values obtained in the evaluation unit are assigned to absolute concentration values, and the confirmation or rejection signal is triggered as a function of the determined gas content on a sorting device, in particular a sorting shunt, such a procedure, defective products are surely sorted out and, on the other hand, adequate products are supplied to further processing such as packaging in containers or shipment.

A device suitable for carrying out such a method may comprise at least one excitation light source arranged at a defined distance from the sensor material provided in the interior of the package, a detection unit fixed to a common holder together with the at least one excitation light source, in particular a photodiode, for the interaction with the fluorescent or luminescent light emitted by the sensor material, and an electronic evaluation unit, and the package containing the sensor material can be placed on a conveyor system moving at a defined constant speed. The essential characteristic features reside in that the sensor material is placed in direct contact with the materials to be measured in a manner separated from the excitation light source and the evaluation unit, and that the package containing the sensor material is placed on a conveyor system moving at a defined constant speed and is arranged to be movable relative to the excitation and detection unit. In this respect, the method is substantially conducted in such a manner that excitation light from the at least one excitation light source impinges on the sensor material, whereupon the latter is excited during passage to emit fluorescent or luminescent light, and the emitted fluorescent or luminescent light is simultaneously detected in the evaluation unit and converted into an electric or electronic signal which is fed to the evaluation unit, and after this the content of the harmful gas contained in the package is detected in a contactless manner and in real time.

In order to obtain safe and consistent and, in particular, reproducible measurement results, the device can substantially be configured such that two LEDs are provided as excitation light sources. By such a device, excitation light will impinge on each individual sensor spot, and each individual package to be examined will be irradiated with excitation light in order to enable the measurement of the gas content in each individual package.

In that the device can be configured such that a plurality of lenses and/or optical filters precede the detection unit, focusing of the fluorescent or luminescent light in the direction to the detection unit is achieved so as to enable the reception of a further harmonized and improved measurement result.

In order to simultaneously ensure that a plurality of light pulses emitted at equal time intervals will impinge on the packages, the device can be further developed such that at least one pulse generator is provided to periodically emit excitation light. By such a device, light pulses are emitted at defined time intervals, in particular between 20 ms and 80 ms, preferably 50 ms, so that excitation light will be fed to each individual sensor spot and at least one fluorescence or luminescence event will be excited by the excitation light on each individual sensor spot in order to reliably enable the examination of each individual package in respect to the gas content in its interior.

In that the evaluation unit can be coupled to a sorting and counting device for the packages, it is furthermore possible to sort out according to their gas content the packages to be examined, wherein packages having gas contents within an admissible range are supplied to further processing, e.g. packaging in containers, and packages having gas contents that are too high or too low will be sorted out. In the same manner, the evaluation unit may include a counting device, by which of not only the overall number of packages, but also the numbers of sorted-out packages and admissible packages, respectively, can be determined.

### BRIEF DESCRIPTION OF THE DRAWINGS

In the following, the invention will be explained in more detail by way of an exemplary embodiment and a drawing. Therein,

FIG. 1 is a schematic illustration of a measurement system according to the invention; and

FIG. 2 is a diagram depicting the measurement results for the content of a gas to be examined in a sample.

### DETAILED DESCRIPTION OF THE INVENTION

### EXAMPLE 1

Oxygen Content in Packages Containing Sausage

A plastic film intended as a cover film for a sausage package, on its surface constituting an inner side after packaging of the sausage product, is coated by sputtering with at least one discreet small spot of a sensor material. To this end, sensor spots are applied to the inner side of the cover-forming film in a contactless manner using a clocked color sputterer (drop on demand). The spots are subjected to drying for two seconds in a hot-air stream while the cover film is forwarded to the packaging device, where food product, e.g. sausage, is introduced into the lower part of the package and a modified atmosphere of the gas to be measured, i.e. O₂ in the present case, is subsequently introduced into the package and hot-sealed. A thus produced package in a first step, immediately upon packaging, is placed on a conveyor device and subjected to contactless measuring of the oxygen content in the interior of the package during conveyance. To this end, a light pulse is emitted every 50 ms from an excitation light source arranged at a distance from the package, which excitation light excites the sensor material provided in the interior of the package to give off fluorescent light as a function of the oxygen content present in the interior of the package. The fluorescent light is focused on a photodiode by a plurality of lenses or mirrors disposed on the same carrier as the excitation light sources. On the photodiode is generated an electric pulse, which, in an electronic system connected to the photodiode, is digitally processed into measurement data relating to the oxygen concentration in the interior of the package.

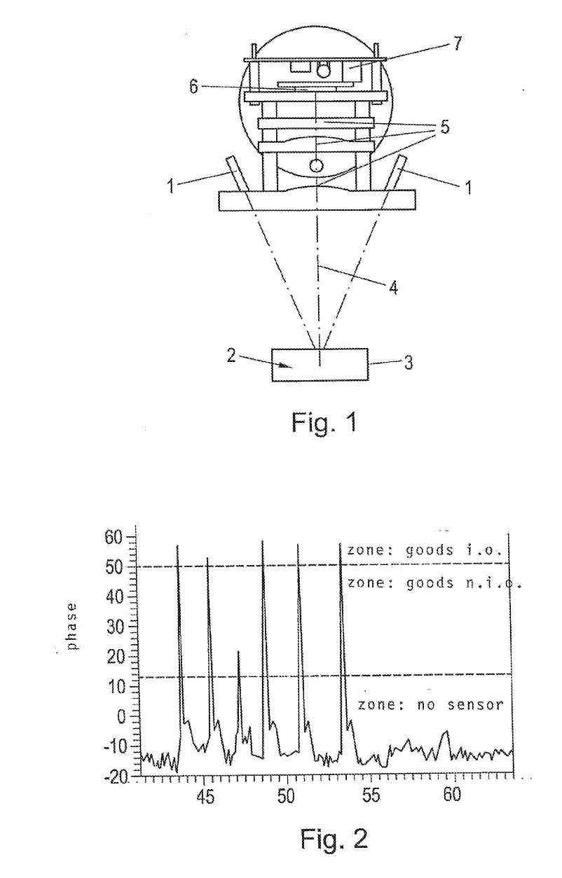

A device of this type is illustrated in FIG. 1. In the device, excitation light pulsed via the excitation light source 1 is allowed to impinge on the sensor 2 disposed in the interior of the schematically illustrated package 3, wherein fluorescent light emitted by the sensor 2 and schematically indicated by 4 impinges on the detection device 6 via a plurality of lenses or mirrors 5. In the detection device 6, the fluorescent light is converted into an electronic signal, which signal is evaluated in the evaluation unit 7 in terms of intensity, and an absolute concentration of the oxygen content to be measured the sample is calculated from the intensity of the signal by comparison with a reference value obtained during a calibration process.

The evaluation unit during the passage of a plurality of packages 3 below the measuring unit, emits a measuring signal each time a sensor 2 has been excited with excitation light 1 to emit fluorescent light. A schematic diagram of the measurement signals is illustrated in FIG. 2. Therein, the measurement value detected in the detection device is assigned to an absolute measurement value by the sensors introduced for the packages 3 having previously been calibrated by the feeding of different gas concentrations and measuring, and the measurement values having been assigned to the respective gas concentrations. In doing so, an oxygen concentration in normal air of about 20% O₂ is set and a calibration between 0.1% O₂ and 20% O₂ is performed, since in the interior of the packages an O₂ content of 0.2% is to be present. In the diagram obtained by such an evaluation unit, a noise is plotted between a phase of −20 and +13, indicating the range in which no sensor is provided, wherein a phase between 13 and 50 represents the range in which the oxygen content in the interior of the sample is too high and the samples are to be rejected, and values having phases of more than 50 are values corresponding to admissible oxygen concentrations in the interior of the packages, thus defining goods that are in good order. From FIG. 2, it is apparent that a sample had too high an oxygen content in the interior of the package, which was subsequently sorted out immediately by the device and discarded.

### EXAMPLE 2

Oxygen Content in a Cleaning and Storing Solution. For Contact Lenses, which is Contained In A Package According to the Invention

A cover film for a glass container intended as a storage container for contact lenses, on its surface facing the cleaning and storing solution after filling and packaging of the contact lenses, is each coated with a discreet spot of a sensor material. The coating is performed by screen printing, and the formed sensor spots are subjected to drying in a hot-air stream prior to being applied to storage container for contact lenses, which contains the storing solution in addition to the contact lenses.

The storing solution in this case is saturated with the gas to be measured, namely oxygen jot the present case. It has a known oxygen content, and the measurement of the oxygen content is effected relative to the ambient oxygen content, wherein differences in the O₂ contents between the interior of the container and the outer side of less than 5% are sufficient to confirm that a sample complies with the predefined criteria, i.e. the oxygen content in the interior of the storage container, in particular the dissolved oxygen content, does not exceed a given limit value of 5%.

Glass containers filled sealed and sealed in this manner are subsequently placed on a conveyor device, and each individual bottle subjected to contactless measuring as described in Example 1, the results are recorded. In doing so, the excitation light source emits the excitation light in a pulsed manner, at a pulse width of 40 ms, in order to safeguard that all of the samples are actually measured. The measuring method itself is identical with that of FIG. 1. A measurement diagram obtained in the test of Example 2 indicates whether the oxygen concentration in the cleaning and storing solution for contact lenses is too high or too low or within the desired range.

In order to receive absolute safety that the produced package is indeed gas-tight, the method can also be carried out in such a manner that immediately after packaging a first measurement is performed as described above, the sample is subsequently pressurized, and after this a second measurement is performed. If the seal of the package is not tight all around, oxygen will in any case be taken up into the interior of the package following the application of pressure, whereupon the second measured value will no longer correspond to the first measured value, thus making clear that the package is leaking, whereupon the product can be immediately sorted out.

It goes without saying that it is not absolutely necessary to calibrate the sensors, but that relative measurements alone will also yield the desired results on whether the content of a package does have gas a combination within an admissible range.

The same arrangement can, for instance, also be used for measuring the gas contents in medicinal articles, pharmaceuticals and electronic components.

### EXAMPLE 3

Measurement of the Oxygen Content in the Argon-filled Interspace of Double-glazed Insulation Windows

In this case, the sensor material is glued or printed to the side of a glass pane facing the interspace between the glass panes and externally excited and read out, as described in Examples 1 or 2. This serves to check whether the filling level with noble gas is sufficiently high.

1. A method for the non-invasive measurement of the gas content of at least one gas selected from O₂, CO₂, amines, nitrogen oxides, sulfur compounds, in transparent packages made of plastic films or other light-permeable materials, with the proviso that said materials are deformable under pressure, in which a sensor material sensitive to the gas content in the interior of the package is excited by excitation light incident thereon to emit a fluorescent or luminescent light, the emitted fluorescent or luminescent light is detected in a detection device interacting with the sensor, whereupon the packaged materials or produced materials are subjected to sorting, and optionally counting, as a function of the gas content determined by an evaluation unit coupled to the detection device, characterized in that the following steps are performed:
a) optionally deep-drawing the package;
b) applying a sensor material to the inner side of a cover for the package, or to the inner side of the package, and optionally drying the sensor material;
c) introducing into the package the materials to be packaged;
d) gas-tightly closing the package by the cover while introducing a modified atmosphere of the gas to be measured;
e) placing the package on a conveying and sorting device;
f) exciting the sensor material by at least one excitation light source arranged at a defined distance from the package;
g) detecting, in a contactless manner, the intensity of the fluorescent or luminescent light emitted by the sensor material as a function of the gas content in the interior of the package, by a detection device arranged at a defined distance from the conveying device during conveying of the package to the sorting device;
h) electrically or electronically converting in the evaluation unit a measurement value detected in the detection device;
i) applying a pressure to the package;
j) performing steps f) to h) again;
k) outputting a confirmation signal or a rejection signal by the evaluation unit; and
l) forwarding the package to further processing or disposal as a function of the gas content in the package calculated by the evaluation unit.

2. The method according to claim 1, wherein the sensor material is applied to the cover of the package by pad printing, screen printing, offset printing, gravure printing, brushing or sputtering, or labeling.

3. The method according to claim 1, wherein a sensor material reacting to O₂, CO₂, NH₃, amines or NO₂ is applied to the cover of the package.

4. The method according to claim 1, wherein the package is evacuated during closure.

5. The method according to claim 1, wherein the measurement of the gas content is performed by the serial excitation and detection of the fluorescent or luminescent light at constant time intervals of 20 ms to 80 ms, an particular 50 ms.

6. The method according to claim 1, wherein measurement values obtained in the evaluation unit are assigned to absolute concentration values, and that the confirmation signal or the rejection signal is triggered as a function of the determined gas content on a sorting device, in particular a sorting shunt.
